(19) United States
(12) Patent Application Publication
(10) Pub. No.: US 2025/0057639 A1
(43) Pub. Date: Feb. 20, 2025

(54) PELVIC STABILIZATION IMPLANTS, METHODS OF USE AND MANUFACTURE

(71) Applicant: SI-Bone Inc., Santa Clara, CA (US)

(72) Inventors: Mary Elizabeth STUART, Santa Clara, CA (US); Zachary LIMA, San Francisco, CA (US); Bret W. SCHNEIDER, San Jose, CA (US); Edward WESTRICK, Gibsonia, PA (US); Conor KLEWENO, Shoreline, WA (US); Charles MOON, Los Angeles, CA (US); John-David BLACK, Richland, WA (US); Reza FIROOZABADI, Mercer Island, WA (US); Bradley DUHON, Castle Rock, CO (US); Francois FOLLINI, Austin, TX (US); Paul M. SAND, Redwood City, CA (US); Scott A. YERBY, Montara, CA (US)

(21) Appl. No.: 18/805,412

(22) Filed: Aug. 14, 2024

Related U.S. Application Data
(60) Provisional application No. 63/519,754, filed on Aug. 15, 2023.

(51) Int. Cl. A61F 2/00; A61B 17/86

(52) U.S. Cl. CPC A61F 2/0045; A61B 17/86

(57) ABSTRACT

Threaded implants, methods of delivering the implants and methods of manufacturing the implants. The threaded implants may be sized, configured and adapted for pelvic stabilization, and may include an elongate body that is optionally monolithic and has a distal end, a proximal end, and a length from the proximal end to the distal end.

### CROSS REFERENCE TO RELATED APPLICATIONS

This application claims the benefit of priority to U.S. Provisional Application No. 63/519,754, titled “PELVIC STABILIZATION IMPLANTS, METHODS OF USE AND MANUFACTURE”, and filed Aug. 15, 2023, the entire disclosure of which is incorporated by reference herein in its entirety for all purposes.

### INCORPORATION BY REFERENCE

All publications and patent applications mentioned in this specification are herein incorporated by reference to the same extent as if each individual publication or patent application was specifically and individually indicated to be incorporated by reference.

This application incorporates by reference herein in their entireties and for all purposes PCT publication WO 2021/108590 A1 and PCT application PCT/US2023/067747.

### BACKGROUND

Some patients may need or benefit from stabilization of at least a portion of their pelvis. For example, without limitation, stabilization following pelvic trauma (e.g., pelvic fractures) and/or bilateral sacro-iliac (“SI”) joint stabilization or fusion may be beneficial or needed. Implants are needed that can be safely implanted across the pelvis, including the SI joints and a sacral corridor, are strong enough to withstand forces thereon following implantation, and will effectively anchor in place in both the ilia and sacrum following implantation.

### SUMMARY

The disclosure herein is related to threaded implants, methods of delivery and manufacture.

One aspect of the disclosure is a threaded implant (“implant”) adapted for pelvic stabilization, including an elongate body (optionally monolithic) having a distal end, a proximal end, and a length from the proximal end to the distal end.

In this aspect, the elongate body optionally includes a plurality of threaded regions including, in a proximal to distal direction, at least two of a proximal multi-lead thread region adjacent the proximal end, a proximal single lead thread region, a central multi-lead thread region, a distal single lead thread region, or a distal multi-lead thread region adjacent the distal end, the plurality of threaded regions including a plurality of threads that are disposed about a shank.

In this aspect, optionally at least one a proximal single lead thread region, a central multi-lead thread region, or a distal single lead thread region optionally includes a plurality of thread interruptions.

In this aspect, optionally at least a portion of a proximal multi-lead thread region and a distal multi-lead thread region include at least one thread that completes at least one revolution about the shank without a thread interruption.

In this aspect, the elongate body optionally further comprises a shank, which optionally defines an inner lumen.

In this aspect, an optional shank optionally includes one or more raised support ribs extending along at least a portion of the elongate body. One or more raised support ribs optionally extend along one or more of a plurality of threaded regions of the elongate body.

In this aspect, a shank optionally includes one or more recesses extending along at least a portion of the elongate body.

In this aspect, one or more optional recesses optionally extend along a portion of each of a plurality of threaded regions of the elongate body.

In this aspect, the elongate body optionally includes a plurality of thread interruptions.

In this aspect, the elongate body optionally includes a plurality of thread interruptions in one or more of a plurality of threaded regions.

In this aspect, the elongate body optionally includes a plurality of thread interruptions in each of a proximal single lead thread region, a central multi-lead thread region, and a distal single lead thread region.

In this aspect, optionally each of a plurality of optional thread interruptions are disposed at one or more recesses of the elongate body.

In this aspect, the elongate body optionally includes a lattice disposed about at least a portion of a shank.

In this aspect, an optional lattice is optionally disposed about at least one of one or more support ribs, or one or more recesses.

In this aspect, a lattice optionally has a thickness at one or more recesses that is greater than a lattice thickness at one or more support ribs.

In this aspect, a plurality of thread interruptions in a central region of the elongate body are optionally helically arranged and helically aligned with a shank recess.

In this aspect, a length of the elongate body is optionally from 140 mm to 180 mm and optionally has a diameter from 8.3 mm to 9.0 mm.

In this aspect, a proximal multi-lead thread region is optionally a dual lead thread region, a central multi-lead thread region is optionally a dual lead thread region, and a distal multi-lead thread region is optionally a dual lead thread region.

In this aspect, a shank, within at least a portion of the shank, optionally does not comprise an opening that creates radial communication between an inner lumen defined by the shank and an ambient space radially outside of the inner lumen. An elongate body may further comprise a plurality of distal cutting flutes adjacent a distal end of the elongate body.

In this aspect, at least one of one or more raised support ribs optionally has a varying height in a radial direction along at least a portion of a length of the at least one support rib.

In this aspect, at least one of one or more raised support ribs optionally has a varying width in a circumferential direction along at least a portion of a length of the at least one support rib.

In this aspect, one or more recesses are optionally curved radially outwardly.

In this aspect, at least one support rib optionally extends from 30 to 100 degrees circumferentially around a shank, and wherein one or more recesses optionally extends from 30 to 100 degrees circumferentially around a shank.

In this aspect, wherein a thread height at a location of one or more support ribs is optionally from 0.8 mm-1.2 mm.

In this aspect, the elongate body may optionally comprise any other suitably combinable feature herein.

One aspect of this disclosure is a threaded implant (“implant”) adapted for pelvic stabilization, including a monolithic threaded elongate body having a distal end, a proximal end, and a length therebetween, the elongate body sized and configured to be laterally implanted such that it extends across first and second SI joints of a subject, and within a sacral corridor while avoiding sacral foramen.

In this aspect, the elongate body may optionally comprise any other suitably combinable feature herein.

One aspect of this disclosure is a method of stabilizing at least a portion of a pelvic with a threaded bone implant, including laterally advancing any of the threaded implants described, claimed, and/or shown herein through a first sacroiliac joint, through a sacral corridor (e.g., an SI corridor, an S2 corridor, or an S3 corridor), and through a contralateral second sacroiliac joint, and leaving the implant implanted across both SI joints and through the sacral corridor.

One aspect of this disclosure is a method of manufacturing a threaded implant, including using an additive manufacturing process to manufacture any of the threaded implants described, claimed, and/or shown herein.

One aspect of this disclosure is a threaded implant (“implant”) adapted for pelvic stabilization. In this aspect, the implant includes an elongate body (optionally monolithic) optionally has a distal end, a proximal end, and a length from the proximal end to the distal end.

In this aspect, the elongate body optionally includes a plurality of threaded regions including, in a proximal to distal direction, a proximal multi-lead thread region adjacent the proximal end, a proximal single lead thread region, a central multi-lead thread region, a distal single lead thread region, and a distal multi-lead thread region adjacent the distal end, the plurality of threaded regions including a plurality of threads that are disposed about a shank, wherein each of the proximal single lead thread region, the central multi-lead thread region, and the distal single lead thread region includes a plurality of thread interruptions.

In this aspect, the shank optionally includes one or more raised support ribs extending along at least a portion of a proximal single lead thread region, a central multi-lead thread region, and a distal single lead thread region.

In this aspect, the shank optionally includes one or more recesses circumferentially adjacent to the one of the one or more raised support ribs.

In this aspect, the elongate body optionally further includes a lattice structure disposed about one or more recesses.

In this aspect, the elongate body may optionally comprise any other suitably combinable feature herein.

In this aspect, a plurality of thread interruptions in each of the proximal single lead thread region, the central multi-lead thread region, and the distal single lead thread region are optionally disposed at the one or more recesses.

In this aspect, a portion of a lattice structure is optionally disposed in at least one thread interruption in each of a proximal single lead thread region, a central multi-lead thread region, and a distal single lead thread region.

In this aspect, one or more raised support ribs are optionally helically arranged about a long axis of the elongate body, and one or more recesses are optionally helically arranged about the long axis.

In this aspect, one or more raised support ribs optionally comprise a plurality of spaced apart raised support ribs.

In this aspect, a lattice structure is optionally disposed about the one or more raised support ribs.

In this aspect, a lattice structure optionally has a thickness at one or more recesses that is greater than a lattice thickness at one or more support ribs.

In this aspect, at least a portion of a proximal multi-lead thread region and a distal multi-lead thread region optionally include at least one thread that completes at least one revolution about the shank without a thread interruption.

In this aspect, one or more raised support ribs are optionally linearly arranged about a long axis of the elongate body.

In this aspect, a length of an elongate body is optionally from 140 mm to 180 mm and optionally has a diameter from 8.3 mm to 9.0 mm.

In this aspect, a proximal multi thread region optionally has a length from 10 mm to 40 mm, a proximal single thread region optionally has a length from 5 mm to 70 mm, a central multi thread region optionally has a length from 10 mm to 80 mm, a distal single thread region optionally has a length from 5 mm to 70 mm, and a distal multi thread region optionally has a length from 10 mm to 40 mm.

In this aspect, a proximal multi-lead thread region is optionally a dual lead thread region, a central multi-lead thread region is optionally a dual lead thread region, and a distal multi-lead thread region is optionally a dual lead thread region.

In this aspect, a central multi-lead thread region optionally does not include a thread that makes a complete revolution about the shank.

In this aspect, a central multi-lead thread region optionally comprises at least three thread interruptions, and optionally at least five thread interruptions.

In this aspect, a proximal single lead thread region optionally does not include a thread that makes a complete revolution about the shank.

In this aspect, a proximal single lead thread region optionally comprises at least three thread interruptions.

In this aspect, a distal single lead thread region optionally does not include a thread that makes a complete revolution about the shank.

In this aspect, a distal single lead thread region optionally comprises at least three thread interruptions.

In this aspect, a proximal multi-lead thread region optionally does not include a thread interruption.

In this aspect, a proximal multi-lead thread region optionally includes a thread that completes at least two revolutions without a thread interruption.

In this aspect, a distal multi-lead thread region optionally does not include a thread interruption.

In this aspect, a distal multi-lead thread region optionally includes a thread that completes at least two revolutions without a thread interruption.

In this aspect, a thread in a proximal single lead thread region, a central multi-lead thread region, and a distal single lead thread region optionally does not complete a revolution about the shank.

In this aspect, a plurality of thread interruptions in a proximal single lead thread region, a central multi-lead thread region, and a distal single lead thread region optionally are helically or linearly arranged.

In this aspect, a shank, in one or more portions of the shank, optionally does not comprise an opening that creates radial communication between an inner lumen defined by the shank and an ambient space radially outside of the inner lumen.

In this aspect, first and second discontinuous and axially spaced support ribs are optionally linearly or helically arranged about a long axis of the elongate body.

In this aspect, one of the one or more raised support ribs optionally has a varying height in a radial direction along at least a portion of a length of the at least one support rib.

In this aspect, one of the one or more raised support ribs optionally has a varying width in a circumferential direction along at least a portion of a length of the one or more raised support ribs.

In this aspect, one or more recesses are optionally curved radially outwardly.

In this aspect, at least one support rib optionally extends from 30 to 100 degrees circumferentially around shank, and wherein one or more recesses optionally extends from 30 to 100 degrees circumferentially around the shank.

In this aspect, a thread height at a location of one or more support ribs is optionally from 0.8 mm-1.2 mm.

In this aspect, the elongate body may optionally comprise any other suitably combinable feature herein.

One aspect of this disclosure is a threaded implant (“implant”) adapted for pelvic stabilization, comprising: a monolithic elongate body having a distal end, a proximal end, and a length from the proximal end to the distal end. In this aspect, the monolithic elongate body optionally includes a plurality of threaded regions including, in a proximal to distal direction, a proximal multi-lead thread region adjacent the proximal end, a proximal single lead thread region, a central multi-lead thread region, a distal single lead thread region, and a distal multi-lead thread region adjacent the distal end, the plurality of threaded regions including a plurality of threads that are disposed about a shank. In this aspect, each of the proximal single lead thread region, the central multi-lead thread region, and the distal single lead thread region optionally includes a plurality of thread interruptions, and wherein at least a portion of each of the proximal multi-lead thread region and the distal multi-lead thread region includes at least one thread that completes at least one revolution about the shank without a thread interruption. In this aspect, a shank may define an inner lumen and includes one or more raised support ribs extending along at least a portion of each of the plurality of threaded regions, one or more recesses extending along at least a portion of each of the plurality of threaded regions, each of the one or more recesses circumferentially adjacent to one of the one or more raised support ribs. In this aspect, the plurality of thread interruptions in each of the proximal single lead thread region, the central multi-lead thread region, and the distal single lead thread region are optionally disposed at the one or more recesses. In this aspect, the elongate body optionally includes a lattice disposed about the one or more support ribs and the one or more recesses in at least a portion of each of the plurality of threaded region, and wherein a portion of the lattice is disposed in at least one thread interruption in each of the proximal single lead thread region, the central multi-lead thread region, and the distal single lead thread region.

In this aspect, the lattice optionally has a thickness at the one or more recesses that is greater than a lattice thickness at the one or more support ribs.

In this aspect, a plurality of thread interruptions in the central region are optionally helically arranged and helically aligned with a shank recess.

In this aspect, a length of the elongate body is optionally from 140 mm to 180 mm and optionally has a diameter from 8.3 mm to 9.0 mm.

In this aspect, at least one of the proximal multi thread region optionally has a length from 10 mm to 40 mm, the proximal single thread region optionally has a length from 5 mm to 70 mm, the central multi thread region optionally has a length from 10 mm to 80 mm, the distal single thread region optionally has a length from 5 mm to 70 mm, or the distal multi thread region optionally has a length from 10 mm to 40 mm.

In this aspect, the proximal multi-lead thread region is optionally a dual lead thread region, the central multi-lead thread region is optionally a dual lead thread region, and the distal multi-lead thread region is optionally a dual lead thread region.

In this aspect, the central multi-lead thread region optionally does not include a thread that makes a complete revolution about the shank.

In this aspect, the central multi-lead thread region optionally comprises at least three thread interruptions, and optionally comprises at least five thread interruptions.

In this aspect, a proximal single lead thread region optionally does not include a thread that makes a complete revolution about the shank.

In this aspect, the proximal single lead thread region optionally comprises at least three thread interruptions.

In this aspect, the distal single lead thread region optionally does not include a thread that makes a complete revolution about the shank.

In this aspect, the distal single lead thread region optionally comprises at least three thread interruptions.

In this aspect, the proximal multi-lead thread region optionally does not include a thread interruption.

In this aspect, the proximal multi-lead thread region optionally includes a thread that completes at least two revolutions without a thread interruption.

In this aspect, the distal multi-lead thread region optionally does not include a thread interruption.

In this aspect, the distal multi-lead thread region optionally includes a thread that completes at least two revolutions without a thread interruption.

In this aspect, a thread in the proximal single lead thread region, the central multi-lead thread region, and the distal single lead thread region optionally does not complete a revolution.

In this aspect, a plurality of thread interruptions in the proximal single lead thread region, the central multi-lead thread region, and the distal single lead thread region are optionally helically or linearly arranged.

In this aspect, the shank, within the central multi-lead thread region, optionally does not comprise an opening that creates radial communication between the inner lumen and an ambient space radially outside of the inner lumen.

In this aspect, the shank, within at least one of the proximal single lead thread region or the distal single lead thread region, optionally does not comprise an opening that creates radial communication between the inner lumen and an ambient space radially outside of the inner lumen.

In this aspect, the shank, within at least one of the proximal multi-lead thread region or the distal multi-lead thread region, optionally does not comprise an opening that creates radial communication between the inner lumen and an ambient space radially outside of the inner lumen.

In this aspect, the shank, within each of a plurality of threaded regions, optionally does not comprise an opening that creates radial communication between the inner lumen and an ambient region radially outside of the respective threaded region.

In this aspect, one or more raised support ribs optionally comprise first and second helical support ribs, and wherein one or more recesses comprise first and second helical recesses, each of the first and second helical recesses circumferentially adjacent to the first and second helical support ribs.

In this aspect, one or more raised support ribs optionally extend continuously in at least the proximal single lead thread region, the central multi-lead thread region, and the distal single lead thread region, interrupted only by threads.

In this aspect, one or more raised support ribs optionally comprise first and second discontinuous and axially spaced support ribs, optionally linearly or helically arranged.

In this aspect, one or more raised support ribs optionally has a varying height in a radial direction along at least a portion of a length of the at least one support rib.

In this aspect, one or more raised support ribs has a varying width in a circumferential direction along at least a portion of a length of the at least one support rib.

In this aspect, one or more shank recesses are optionally curved radially outwardly.

In this aspect, at least one support rib optionally extends from 30 to 100 degrees circumferentially around shank, and wherein one or more recesses optionally extends from 30 to 100 degrees circumferentially around the shank.

In this aspect, the elongate body may optionally comprise any other suitably combinable feature herein.

### BRIEF DESCRIPTION OF FIGURES

FIG. 1 is an exemplary threaded implant for pelvic stabilization.

FIG. 2 shows an exemplary threaded implant implanted for pelvic stabilization.

FIG. 3A illustrates a proximal portion of an exemplary threaded implant.

FIG. 3B illustrates a distal portion of an exemplary threaded implant.

FIGS. 4A, 4B and 4C illustrate sections of an exemplary threaded implant.

FIGS. 5A, 5B and 5C illustrate sections of an exemplary threaded implant.

FIG. 6 is an exemplary threaded implant for pelvic stabilization.

FIG. 7 is an exemplary threaded implant for pelvic stabilization.

FIG. 8A illustrates a distal portion of an exemplary threaded implant.

FIG. 8B illustrates a proximal portion of an exemplary threaded implant.

FIG. 9 is an end view of an exemplary threaded implant for pelvic stabilization.

FIGS. 10A, 10B, 10C and 10D are an exemplary threaded implant for pelvic stabilization.

FIG. 11 illustrates exemplary dimensions herein.

FIG. 12 is an exemplary threaded implant for pelvic stabilization.

FIGS. 13A, 13B, and 13C illustrate exemplary raised support structures.

FIG. 14 illustrates an exemplary raised support structure.

FIG. 15 illustrates an exemplary raised support structure.

### DETAILED DESCRIPTION

One aspect of the disclosure is related to threaded implants adapted for lateral implantation and sized, configured and arranged to stabilize at least a portion of a pelvis, optionally for pelvic trauma and/or stabilizing the SI joints. Threaded implants herein are sized and configured for anchoring and stability once implanted.

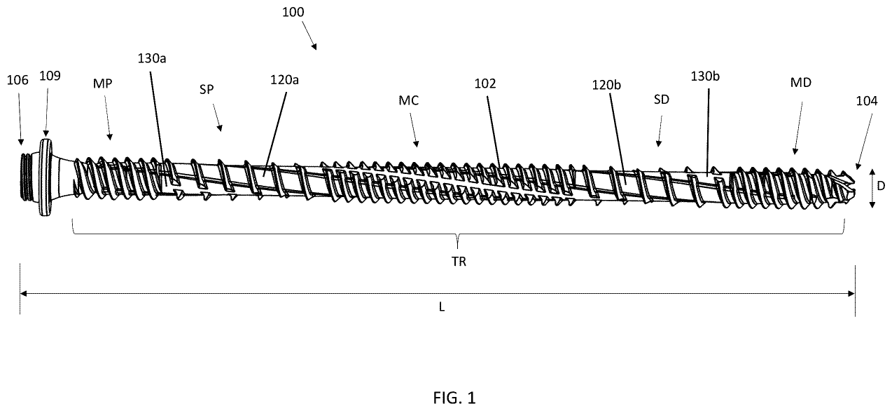

FIG. 1 is a side view of an exemplary threaded implant 100 that is sized and configured for lateral implantation and to stabilize at least a portion of a pelvis, optionally for pelvic trauma and/or stabilizing the SI joints. Implant 100 includes an elongate body 102, which may be a monolithic elongate body optionally formed using an additive manufacturing process such as 3-D printing. A monolithic elongate body in this example would exclude separate washer 109. Elongate body 102 includes a distal end 104, a proximal end 106, and length “L” from proximal end 106 to distal end 104, as shown. Elongate body 102 is an example of a body that is sized and configured to be laterally implanted such that it extends across first and second SI joints of a subject, and within a sacral corridor while avoiding sacral foramina, which is described in additional detail below.

The implant length L is long enough so that the implant extends across both SI joints when implanted laterally, yet short enough to prevent the implant from extending laterally beyond the iliac walls to an extent that it causes significant tissue damage or discomfort.

The length L (and of any of the threaded implants herein) may be, inclusive, from 140 mm to 180 mm, such as from 150 mm to 175 mm or from 160 mm to 170 mm. The ranges herein, including for the length of the threaded implants herein, inherently include all possible subranges within ranges (e.g., 155-165, 154-169), as well as discrete length dimensions in the ranges (e.g., 155; 156; 157; 167; etc.).

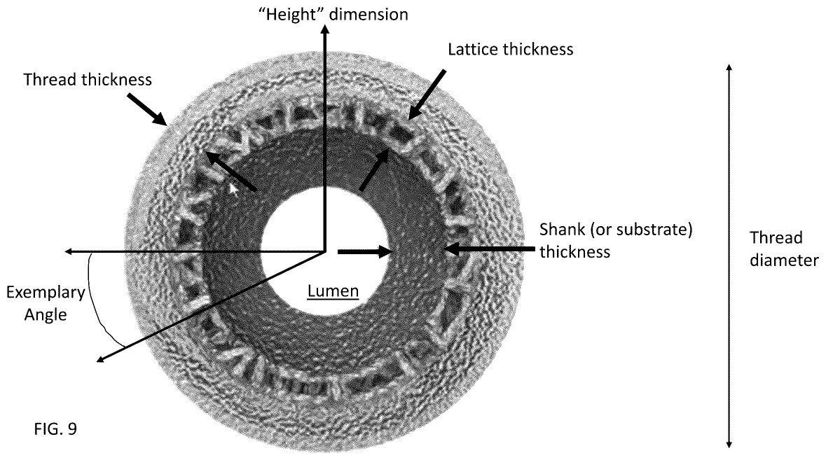

Elongate body 102 has a thread diameter “D,” (labeled in FIG. 1 and referenced generally in FIG. 9) which refers to a major diameter of at least one thread along at least a portion of length L, and excludes the optional threads shown at proximal end 106. The diameter “D” of body 102 in this context refers to a major diameter along more than half of implant threaded region, such as in threaded region “TR” labeled in FIG. 1. At least one thread may have a thread height that is slightly greater, for example, in the MP region, yet the elongate body may still be considered to have a diameter D. The diameter may be, inclusive and for example, from 8.3 mm to 9.0 mm, from 8.4 mm to 9.0 mm, from 8.5 mm to 9.0 mm, or 8.6 mm to 9.0 mm. Diameter dimensions herein are measured through a longitudinal axis of the implant elongate body.

As used herein, an implant threaded region “TR” may comprise a plurality of threaded regions, each having at least one characteristic that is different than at least one other threaded region in the plurality of threaded regions. In the example in FIG. 1, the threaded region TR comprises five threaded regions as shown, as long as the length of the threaded region TR. In alternative examples, there may be axial spacing between any axially adjacent thread region. Implants herein may include a threaded region TR that essentially includes a single threaded region (rather than a plurality of threaded regions), although such implants may not provide as many benefits as other implants herein, such as not being able to anchor as well in the pelvis. Additionally, threaded regions that include a plurality of threaded regions may include two, three, four, five, six, seven, eight or more separate threaded regions each having at least one characteristic (e.g., the number of leads) that is different from at least one of the other thread regions in the plurality of thread regions. Implant 100 in FIG. 1 is an example of an implant where each of the plurality of thread regions includes at least one characteristic that is different than at least one characteristic of one or both adjacent thread regions.

The threaded implants herein may include thread regions with one or more characteristics or features that provide advantages based on where the thread region is situated when implanted, examples of which are provided below.

Elongate body 102 is an example of an elongate body that has a multi thread proximal region MP, a single thread proximal region SP, a multi thread central region MC (positioned to be at least partially implanted in a sacral corridor), a single thread distal region SD, and multi thread distal region MD, as shown. Any of the MP's herein may be, inclusive and for example, from 10 mm to 40 mm long. Any of the SP's herein may be, inclusive and for example, from 5 mm to 70 mm long (including any whole number therein). Any of the MC's herein may be, inclusive and for example, from 10 mm to 80 mm long (including any whole number therein). Any of the SD's herein may be, inclusive and for example, from 5 mm to 70 mm long (including any whole number therein). Any of the MD's herein may be, inclusive and for example, from 10 mm to 40 mm long (including any whole number therein). In the example in FIG. 1, each of the multi thread regions is a dual thread region, but one or more of the multi thread regions could optionally be more than two, such as three leads.

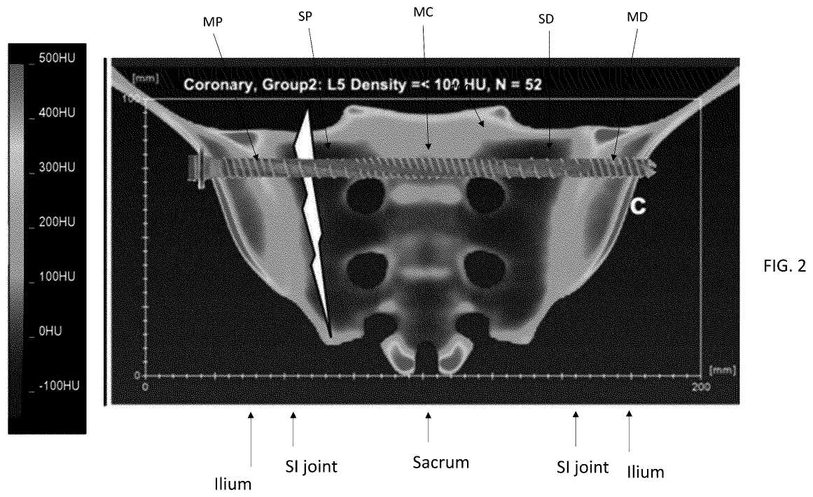

FIG. 2 illustrates an exemplary implant position of threaded implant 100 following lateral implantation across the pelvis including through both SI joints. It is understood that any of the threaded implants herein may be implanted in the same position as is shown in FIG. 2, even if the implant does not include the features of implant 100 and/or if it includes features not described with respect to implant 100. The threaded implant shown in FIG. 2 has an elongate body with a dual-single-dual-single-dual thread arrangement, an example of which is shown in FIG. 1. Merely exemplary and nonlimiting lengths of threaded regions for the implant in FIG. 2 are as follows: DP may be about 15 mm; SP may be about 30 mm; DC may be about 55 mm; SD may be about 30 mm; DD may be about 25 mm. Any of the elongate bodies herein may have an MD that is longer than an MP (optionally slightly longer), of which FIGS. 1 and 2 are examples. The multi lead threads in MP and MD enhance anchoring in the left and right ilia (illustrated in FIG. 2), while the multi lead threads in MC enhances anchoring in the central region of the sacrum (also illustrated in FIG. 2). The single threads in SP and SD are positioned and sized to be disposed across the SI joints (illustrated in FIG. 2), wherein the single threads allow more space on the implant for more porous lattice structure (described below) in the sacral alar region, which promotes relatively more ingrowth, on-growth or through-growth at the locations near the SI joints where needed. The implants herein may thus include a plurality of threaded regions, wherein the number of threads enhances anchoring, and/or facilitates enhanced boney growth (e.g., by having fewer threads and allowing for additional growth features, such as relatively more lattice structures) based on where the threaded region will be positioned when the threaded implant is implanted in the pelvis.

The elongate bodies of the implants herein may include a shank (which may also be referred to herein as a substrate), from which one or more threads extend radially outward. Shanks and threads may be monolithically formed, such as with an additive manufacturing technique. For additional strength, any of the shanks herein may include one or more raised support ribs along at least a portion of the elongate body. Elongate body 102 of implant 100 in FIG. 1 includes first and second support ribs 120a and 120b, which in this example have optional helical or spiral configurations about and relative to a longitudinal axis (“LA”) of elongate body 102. Support ribs herein are regions of an elongate body with a greater height (measured radially from a long axis in a direction orthogonal to the long axis) than a height of an adjacent shank portion, creating a raised region of the shank (relative to the height of the adjacent shank portion) for added strength.

The raised support ribs herein are generally shown and described as being longer than they are wide (i.e., elongate), but in alternative designs they may have a width (measured around the implant) greater than an axial length. For example, a support rib may have a relatively short length (axially) and extend around the shank a distance greater than its length.

Shanks herein that include one or more raised support structures (e.g. support ribs) include an adjacent shank region with relatively less height, which herein are referred to as shank or substrate recesses. It is understood that the term recess as used herein does not require a portion of a shank from which material has been removed from a starting material during manufacturing, but merely that it has less height than an adjacent raised region. For example, additively manufactured threaded implants herein may include forming raised shank regions that have a greater height than an adjacent shank region, wherein the adjacent lower shank region is recessed relative to the raised region. Any recess herein may also be referred to herein as a shank recess or substrate recess, and vice versa.

Implant 100 includes substrate recesses 130a and 130b, which in this example also have optional helical configurations about elongate body 102, each of which is circumferentially adjacent to a raised support region (in this case support rib). Exemplary recesses are shown in FIGS. 1, 2, 3A, 3B, 4A, 4B, 4C, 5A, 5B, 5C, 6, 7, 8A and 8B.

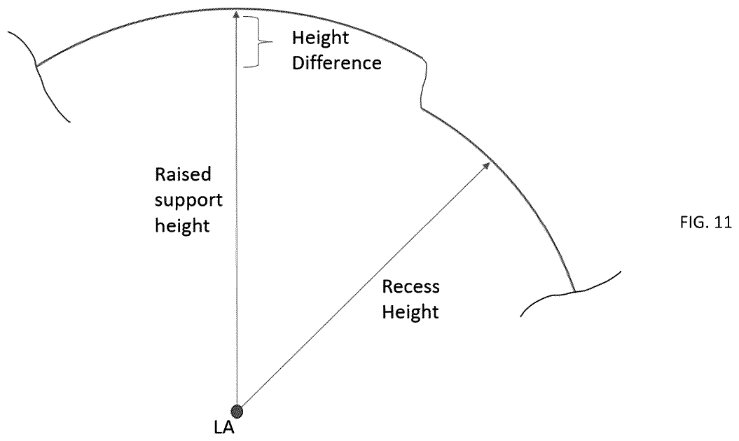

FIG. 11 is meant to illustrate an end view difference in Height dimension between any of the shank recesses herein and an adjacent shank raised support region. In the illustrative FIG. 11, threads are not shown for clarity nor are additional optional implant features (e.g., an inner lumen and/or lattice structure). The height dimensions are measured relative to the illustrative long axis “LA,” in a direction orthogonally to the LA, as shown.

Any of the raised supports herein (e.g., supports ribs) may be from 0.1 mm to 2 mm radially higher (taller) than adjacent substrate recessed regions, with a general height difference between circumferentially adjacent sections shown in FIG. 11. The raised supports may also have a radial thickness or lattice thickness (measured from an inner aspect to an outer aspect orthogonal relative to a long axis, as shown generally in FIG. 9) greater than radial thicknesses of adjacent recessed shank regions.

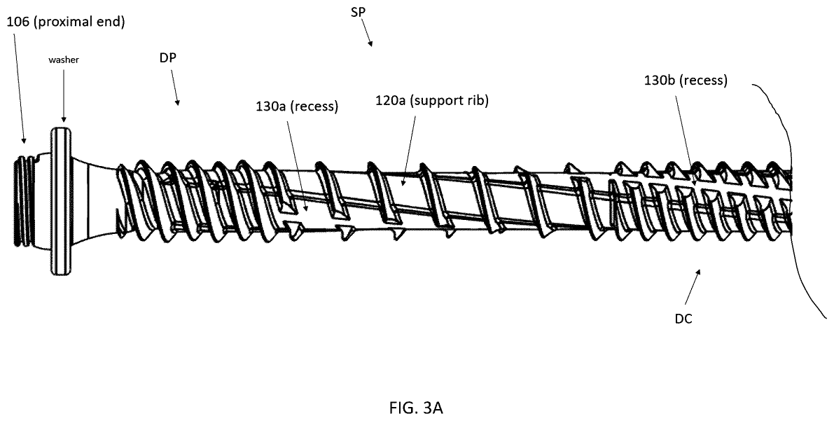

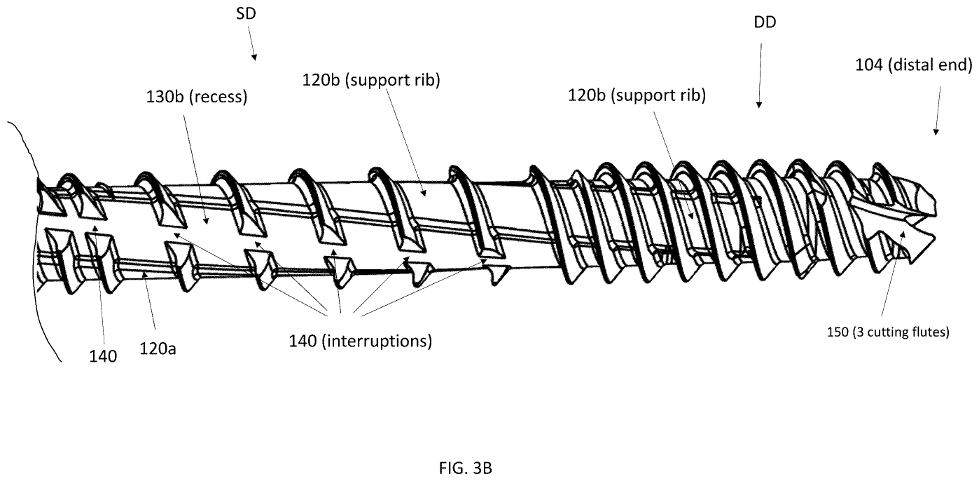

FIG. 3A shows a side view of a proximal portion of elongate body 102, and FIG. 3B shows a side view of a distal portion of the elongate body 102 (about half of the implant shown in each of FIGS. 3A and 3B), showing aspects of exemplary elongate body 102 including first and second helical support ribs and first and second adjacent helical substrate recesses. As shown, the support ribs and the substrate recesses in this example extend from the dual thread proximal region DP to the dual thread distal region DD, including regions SP, DC and SD.

Any of the implants that include a raised support structure may include at least one raised support structure that extend through one or more implant thread regions, and optionally at least through at least a portion of all thread regions of the implant. Raised support structures herein may optionally have a length such that they continuously extend (i.e., without a break or discontinuity) through all of an implants thread regions, even if the raised support structure does not extend all the way to the proximal and/or distal ends of the implant thread region (of which raised ribs 120a and 120b are examples).

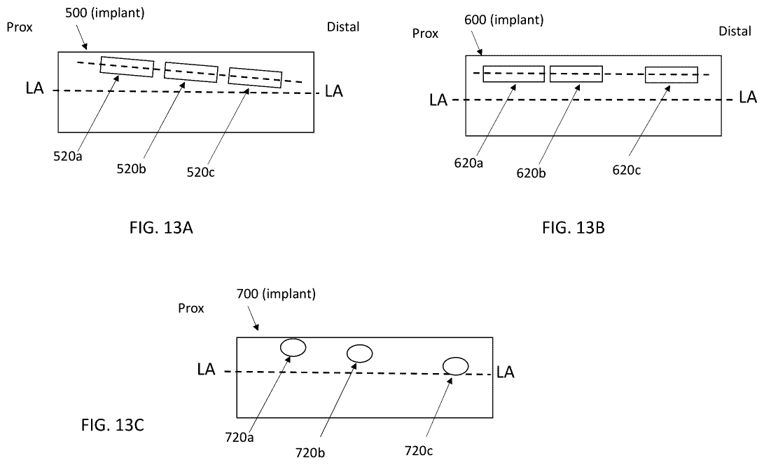

Alternatively, any of the implants herein may include a plurality of raised support structures that are spaced along the elongate body in one or more thread regions. By way of example only, FIG. 13A represents a generalized implant 500 with a long axis and proximal and distal ends (prox and distal). Implant 500 includes a plurality of raised support structures 520a, 520b and 520c that are spaced along at least a portion of the length of the implant. In this example, the support structures 520a, 520b and 520c are helically arranged about LA. In this example, the support structures are spaced apart by the same distance or substantially the same distance. The implant may include more or fewer support structures than shown, and implant 500 may include any implant feature herein. By way of an additional example, FIG. 13B represents a generalized implant 600 with a long axis and proximal and distal ends (prox and distal). Implant 600 includes a plurality of raised support structures 620a, 620b and 620c that are spaced along at least a portion of the length of the implant. In this example, the support structures 620a, 620b and 620c are linearly arranged about LA along an axis that is parallel to LA. In this example, at least two of the support structures are spaced apart a distance that is different than a distance two other support structures are spaced apart, as shown. The implant may include more or fewer support structures than shown, and implant 600 may include any implant feature herein. By way of an additional example, FIG. 13C represents a generalized implant 700 with a long axis and proximal and distal ends (prox and distal). Implant 700 includes a plurality of raised support structures 720a, 720b and 720c that are spaced along at least a portion of the length of the implant. In this example, the support structures 720a, 720b and 720c are generally helically arranged about LA. In this example, at least two of the support structures are spaced apart a distance that is different than a distance two other support structures are spaced apart, as shown. The implant may include more or fewer support structures than shown, and implant 600 may include any implant feature herein.

In alternative implants, a plurality of raised support structures may not be regularly arranged about an axis for example, but may have more of an irregular distribution while still providing structural support along at least portion of the implant. In an alternative implant, a plurality of raised support structures may have a random arrangement while still providing structural support along at least portion of the implant.

Any of the support structures herein, whether continuous, or a support structure that comprises a plurality of individual spaced support structures such as those shown in FIGS. 13A-13C, may extend along 5% to 99% of the elongate body length L, such as 5% to 95% of the length L, or along 60% to 95% of the elongate body length L, or along 65% to 95% of the elongate body length L, or along 70% to 95% of the elongate body length L, or along 75% to 95% of the elongate body length L. In the example in FIG. 1, the support ribs extend along about 80% to about 90% of the elongate body length L.

Any of the support structures herein, whether continuous, or a support structure that comprises a plurality of individual spaced support structures such as those shown in FIGS. 13A-13C, may extend along 5% to 99% of the length of the threaded region TR, such as 5% to 95% of the length of TR, or along 50% to 95% of the length of TR, or along 65% to 95% of the length of TR, or along 70% to 95% of the length of TR, or along 75% to 95% of the length of TR. In the example in FIG. 1, each of the plurality of support ribs are examples of continuous raised support structures that extend along about 80% to about 98% of the length of TR.

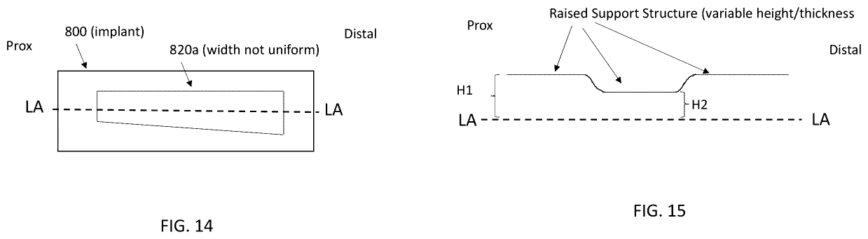

In alternative implants, one or more raised support structures may have a width (measured generally around the elongate body) that is not uniform or constant along the length of the support structure, a mere example of which is shown in the top view of implant 800 in FIG. 14. Implant 800 includes support structure 820a, for which the width is not uniform as shown. Implant 800 may include additional support structures, and implant 800 may include any implant feature herein. That is, any of the implants herein may include at least one raised support structure with a width that is not constant along its length.

In alternative implants, one or more raised support structures may have a height or thickness dimension that is not constant or uniform along its length, a mere illustrative of which is shown in a side view in FIG. 15 in which a portion of an exemplary support structure is shown. Threads and other optional implant features are not at all shown in FIG. 15 for clarity. As shown, a first height (H1) of the exemplary raised support is greater than a second height (H2) along the length of the implant. The height at the distal end of the figure may be the same as H1 or a different H3. Any exemplary reason why a support structure height (or thickness) may vary is that there may be a trade off between support needed and desired lattice structure to promote in-growth, on-growth, or through growth. For example, support structures may have thinner dimensions (e.g., H2 as shown in FIG. 15) in regions where less stress might be expected on the implant following implantation, and optionally where it may be more desirable to have additional lattice structure in that region for better anchoring. Additionally, support structures may have thicker dimensions (e.g., H1 as shown in FIG. 15) in regions where more stress might be expected on the implant following implantation. Any of the support structures herein (whether they are continuous or comprise a plurality of space structures) may have non-constant heights. Additionally, in implants that include a plurality of axially spaced support structures, such as shown in FIGS. 13A-13C, individual support structures may have different heights. For example, in FIG. 13A, support structure 520a may have a height that is greater than the height of support structure 520b.

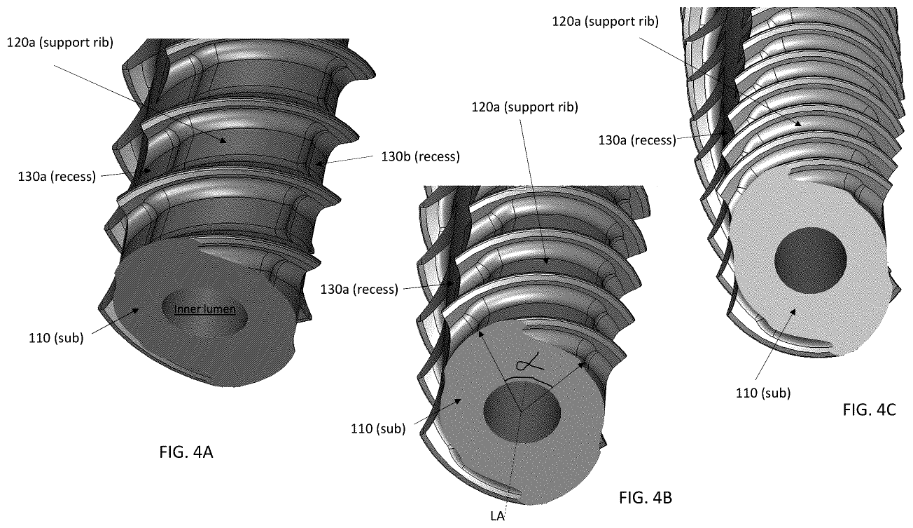

FIGS. 4A, 4B, and 4C illustrate elongate body 102 when cut orthogonally to long axis LA in the threaded section, including support structure 120a (in this example a support rib) and recesses 130a and 130b. Long axis “LA” of the elongate body is labeled in FIG. 4B, but is understood to apply to any of the elongate bodies herein. Support ribs and adjacent substrate recesses herein can be described as circumferentially extending around the elongate body at an angle or in a range of angles. Angle alpha shown in FIG. 4B (relative to long axis LA) illustrates the circumferential extent or span of support rib 120a, at least in this cross section. Additionally, FIG. 9 represents an exemplary angle that any of the implant features herein may circumferentially extend around the elongate body (e.g., threads, raised support structures, recesses, etc.). Any of the support ribs herein may optionally extend from 30 to 135 degrees circumferentially around the shank 110, relative to a long axis of the elongate body. Any of the substrate recesses may optionally extend from 10 to 135 degrees circumferentially around the shank, relative to a long axis of the elongate body. First and second support ribs of a shank may extend the same angle circumferentially around the shank, relative to a long axis, an example of which is shown in elongate body 102. First and second substrate recesses may extend the same angle circumferentially around the shank, relative to a long axis, an example of which is shown in elongate body 102. Support ribs and recesses may all extend the same or substantially the same angle circumferentially around the elongate body.

FIG. 14 described an exemplary implant 800 with at least one raised support structure with a width dimension that is not constant along its length. This is alternatively stated, with reference to the “angle” shown in FIGS. 4B and 9, as a support structure with a circumferentially spanning angle that is not constant or uniform along the axial length of the support structure. For example, the proximal end of the raised support structure 820a in FIG. 14 would extend an angle around the elongate body that is less than an angle of the distal end of raised support structure 820a. Any of the implants herein may include one or more raised support structures that, like, support structure 820a, does not extend a constant angle around the elongate body along its entire length. The width of the support structure(s) may vary in any way that is desired to provide the desired strength and/or bony growth.

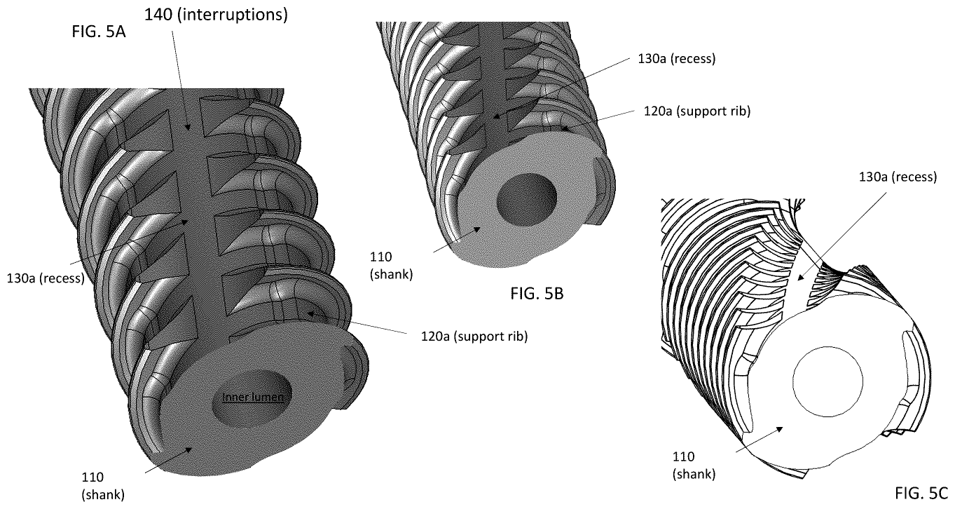

FIGS. 5A, 5B, and 5C show section views of elongate body 102, but rotated relative to FIGS. 4A-4C to illustrate more clearly shank recesses 130a and 130b. The entire disclosure herein related to FIGS. 4A-4C, including the disclosure related to ribs and recesses extending circumferentially around the elongate body applies to FIGS. 5A-5C and is incorporated fully into FIGS. 5A-5C and the text description thereof.

As shown in the examples of FIGS. 4A-5C, the at least one support rib and the at least one recess have radially outer surfaces that are outwardly curved (convex), and optionally have a generally smooth outer curve. As is described below, even in examples wherein the elongate body includes lattice sections extending radially outward from support ribs and/or recesses of a shank, the at least one support rib and the at least one substrate recess of the shank are optionally considered to have outer regions that have surfaces that are outwardly curved.

In any of the examples herein, at least one support structure (e.g., rib) may not extend all the way to the distal end of the elongate body, of which elongate body 102 is an example. For example, one or more support structures may have distal ends that are from 5 mm to 20 mm away from the elongate body distal end (measured axially along the LA of the implant), optionally 5 mm to 15 mm away from the distal end.

To increase the strength of the elongate body, any of the elongate bodies herein may be free of fenestrations that extend all the way through the substrate. The entire disclosure of fenestrations in WO 2021/108590 A1 is incorporated by reference herein. Elongate body 102 is an example of an elongate body that is free of fenestrations. It is of note that elongate bodies herein may optionally include one or more cutting flutes, such as the three cutting flutes labeled in FIG. 3B, which are not considered to be fenestrations as used herein. The elongate bodies herein may thus have distal cutting flutes and can still be considered to be free of fenestrations that extend from an outside of the substrate and all the way radially through the substrate to an inner or central lumen. An inner lumen or channel of elongate body 102 is labeled in FIGS. 4A and 5A, although any of the elongate bodies herein may optionally not include a central lumen, in which case they are considered to have a solid inner core.

In some implants that have one or more threaded implant regions, one or more (including all) of the threaded regions may be free of fenestrations. Any combination of threaded regions, however, may include one or more fenestrations through the elongate body. For example, regions where stresses may be expected to be relatively lower may include one or more fenestrations that extend from outside the elongate body to a central lumen.

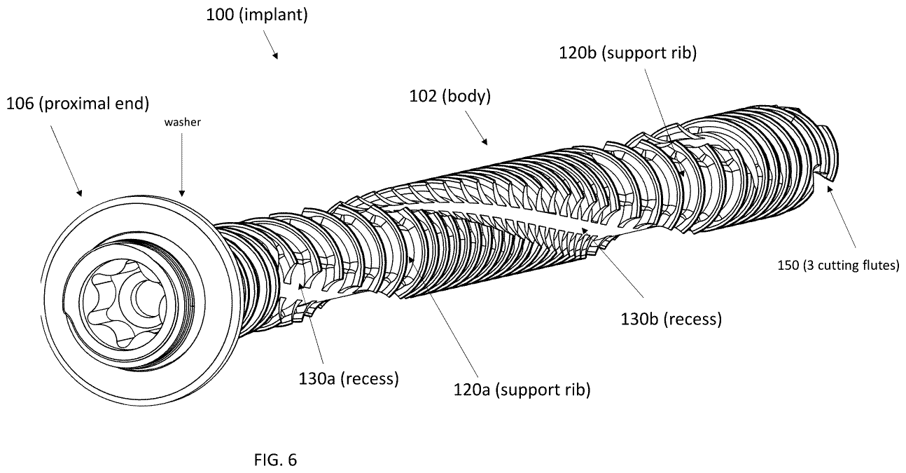

FIG. 6 illustrate a proximal perspective view of implant 100, including elongate body 102. Any and all disclosure herein related to elongate bodies (including in the figures and claims) is optionally integrated into the example and description in FIG. 6.

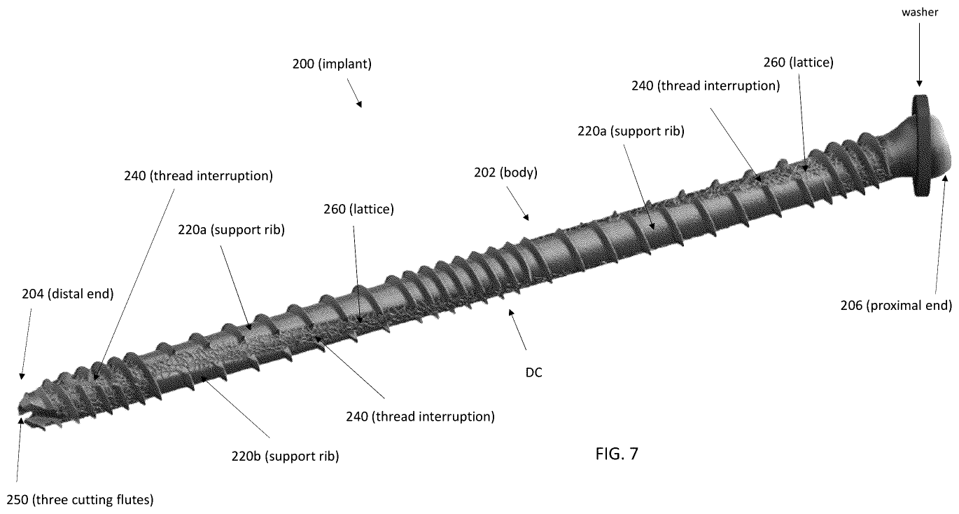

As referenced herein, the threaded implants need to be strong enough to withstand forces applied along the implant. At least a portion of the implants herein may include a lattice structure to promote bony in-growth, on-growth and through-growth to increase stabilization after implantation. FIG. 7 illustrates an exemplary implant 200 that is sized and configured for lateral implantation to stabilize at least a portion of a pelvis, optionally for pelvic trauma and/or stabilizing the SI joints. Any of the disclosure related to FIG. 7 is optionally incorporated into any of the other threaded implants herein, and vice versa (including shown in the figures and/or recited in claims). Implant 200 includes an elongate body 202, proximal end 206, distal end 204, distal cutting flutes 250, support structures in the form of elongate support ribs 220a and 220b (which in this example are each continuous and helical) and lattice or lattice structure 260 disposed about at least a portion of the shank of elongate body 202. Lattice 260, in this example, is disposed about and extends axially through the optional five threaded regions, as shown. In this example, and in a portion of the implant, lattice 260 is shown extending radially from shank recesses but not support ribs 220a and 220b. In alternatives, the lattice (e.g., lattice 260) may also extend radially from support ribs and recesses, in which case the lattice of the elongate body extends between the threads along at least most of the length of the elongate body, including in all threaded regions. The lattice herein may be referred to as a porous lattice comprising a network of interconnected struts that extends radially outward from the substrate. The disclosure related to lattice structures described in WO 2021/108590 A1 is incorporated by reference herein in its entire for all purposes, including exemplary methods of manufacturing.

In the example of FIG. 7, the raised support structures 220a and 220b extend through a proximal single-thread region, a central multi-thread region, and a distal single thread region, as shown. In this merely exemplary implant, the support structure does not extend (or only minimally extend) in the proximal and distal multi-thread region, although in alternative designs they may extend one or both end thread regions. In the example of FIG. 7, the lattice is disposed about all or substantially all of the shank in the proximal and distal multi-thread regions, as shown.

In alternative examples, not all threaded regions may include a lattice structure upon the shank. For example, only a subset of threaded regions may include a lattice structure.

There may be applications where strength of the elongate body from the shank is more important than including lattice structure in one or more regions of the elongate body, and, for example only, one or more threaded regions may be free of lattice (along a least a portion of that threaded region's length) to allow the shank to have a greater height in those regions. Additionally, for any combination of threaded regions, lattice structure may be disposed along at least some portion of the length of the shank of the threaded region, but optionally not the entire length of the shank of the threaded region, in which case there may be axially spaced sections of lattice within a single thread region.

Any of the lattice section(s) herein may be formed monolithically upon the shank, such as using an additive manufacturing process (e.g., 3D printing).

The lattices (lattice structures) herein allow for boney on-growth or bone incorporation generally, which is important for implant stabilization. In the example shown in FIG. 7, the threaded regions that are single thread (i.e., SP and SD) provide a greater amount of relative shank surface area (due to a single thread rather than multi-thread) to support lattice structure, which promotes relatively more bony integration with the implant. FIG. 7 also includes an optional additional feature to further promote bony integration in one or more regions of the implant, which includes one or more thread interruptions 240 along at least a portion of one or more threads. Thread interruptions are also shown in FIGS. 3B and 5A as thread interruptions 140. A thread interruption (i.e., a break or discontinuity in a thread at that location) provides more substrate surface area upon which lattice can be formed, enhancing even further the integration with bone.

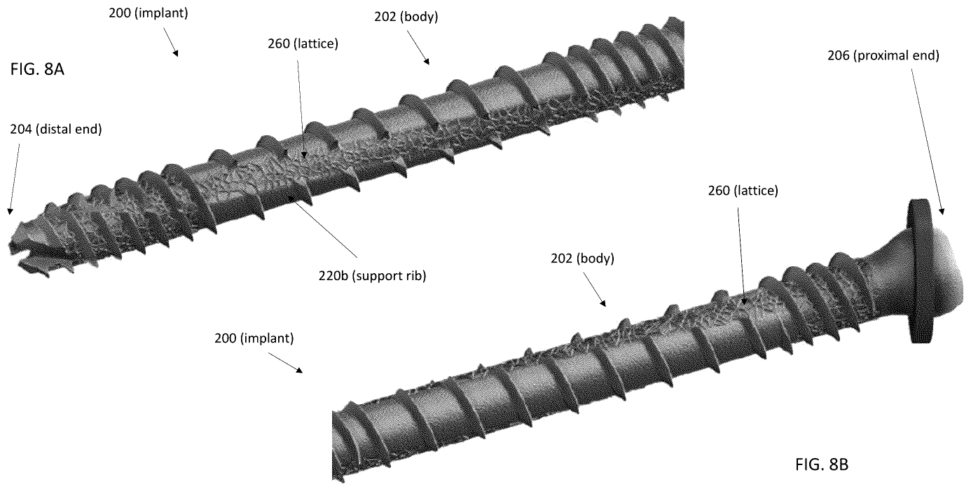

Elongate body 102 and elongate body 202, as shown, are examples of threaded implants that include a plurality of thread interruptions along at least a portion of one or more thread regions. In this example, elongate body 202 includes a plurality of thread interruptions in at least a portion of each of a single lead proximal region, a multi-lead central region, and a single lead distal region. In the particular example in FIG. 7, the plurality of thread interruptions are examples of thread interruptions that are helically arranged, as shown. The helically arranged interruptions in this example include a plurality of thread sections that do not extend a complete revolution about the long axis between the start and stop of the thread sections, which in this example forms a helical arrangement of the thread interruption. In alternative designs, a plurality of thread interruptions may have a linear arrangement along the shank length, the linear arrangement extending parallel or generally parallel to the long axis. FIGS. 8A and 8B illustrate close up views of implant 200 shown in FIG. 7

While there may be advantages for having some thread regions with many thread interruptions, implants herein may have one or more thread interruptions along any portion of the elongate body length (or in some alternatives, no thread interruptions). The thread interruptions may not extend into or fully through a multi lead proximal region and/or may not extend into or fully through a multi thread distal region.

In alternative examples, a plurality of thread interruptions may not be “continuous” and do not have a helical or linear arrangement, in that the interruptions may occur every other turn, for example only. In these alternatives, a thread section would make a complete revolution between the start and stop of that particular section.

Any of the thread interruptions herein can, similar to the raised support structures recesses herein, be described as extending circumferentially around the shank at an angle or a range of angles. The angle can be measured in the same manner as is angle alpha shown in FIG. 4B and referenced in FIG. 9, but instead applicable to the angle of one or more thread interruptions. Any of the thread interruptions may extend from 5 degrees to 90 degrees circumferentially around the shank (or more), relative to a long axis LA of the elongate body.

Implant body 202 in FIG. 7 includes an optional additional feature to further promote bony integration in one or more regions of the implant. As discussed above, implant includes thread interruptions 240, and in this example a plurality of thread interruption are disposed or situated at or within one or more shank recesses. By situating thread interruptions at and/or within one or more recesses (compared to interruptions in the raised support surfaces), even more potential space/volume is created for lattice structure at the location of the interruption due to the shorter height of the shank in the recess, further enhancing bony integration with the implant. Any of the elongate bodies herein may include helically arranged thread interruptions that are aligned with a helical recess, examples of which are shown FIGS. 1-8B. In alternative examples, any of the elongate bodies herein may include linearly arranged thread interruptions that are aligned with a linear recess (both parallel to a long axis of the elongate body).

Any of the disclosure in WO 2021/108590 A1 related to porous lattice sections, including their formation, is expressly included herein and is optionally integrated into any of the lattice or lattice structure herein.

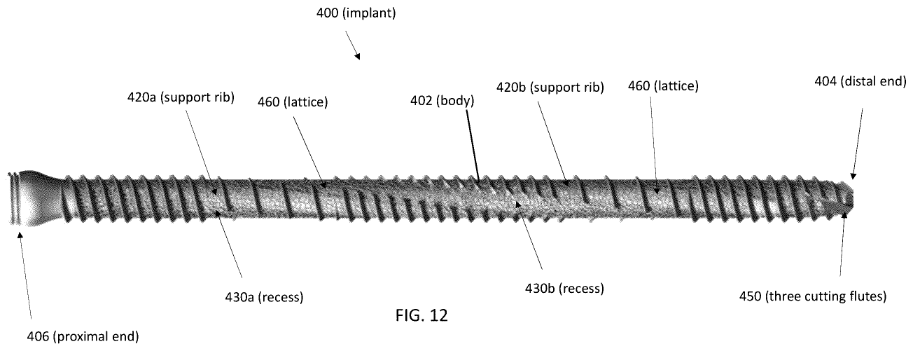

FIG. 12 Illustrates an exemplary threaded implant 400, and which may include any feature of any other implant described, shown or claimed herein. Implant 400 is similar to implant 200 in FIG. 7, and also includes lattice upon raised support structures 420a and 420b. Additionally, raised support structures also extend into the multi-lead distal and proximal regions, as shown. Implant includes a plurality of thread regions (in this example, five), a plurality of thread interruptions at recess locations, and wherein helically arranged thread interruptions are aligned with helical recesses, as shown. Implant 400 is shown free of fenestrations in all thread region, and includes a plurality of cutting flutes.

Implant 400 includes lattice structure on both recesses and raised support regions. In this example, the lattice may inherently have a greater height in the recessed region(s) than in the raised support structure region due to the radially recessed nature of the recess(es) relative to the raised support structure. This may be the case where the height of the lattice is constant or substantially constant in both the recessed region(s) and the raised support structure(s).

FIG. 9 illustrates an end view (looking along the long axis) reference cross section that may be used in the description any of the threaded implants and elongate bodies herein. FIG. 9 shows a shank (defining an inner lumen in this example), a lattice formed upon the shank (optionally monolithically formed with the shank, such as via additive manufacturing, such as 3-D printing) and extending radially outward from the shank, and a portion of a thread formed upon the shank and extending radially outward from the shank (optionally monolithically formed with the shank, such as via additive manufacturing, such as 3-D printing). Thickness dimensions are illustrated, measured from an inner aspect to an outer aspect of the structure. In any of the examples herein, a thread thickness may be 0.8 mm-1.2 mm, optionally 0.8 mm, 0.9 mm, 1 mm, 1.1 mm, or 1.2 mm, measured from a minor dimension (root), as illustrated in FIG. 9. A constant thread height may optionally extend in a proximal single thread region, a central multi thread region, a distal single thread region, and at least a portion of a distal multi thread region (such as any of those thread sections herein). With any of the elongate bodies herein, one of the threads in a proximal multi thread region may have a greater thread height than a different thread in the dual multi proximal region. With any of the examples herein, a substrate thickness may be from 0.5 mm to 2.0 mm, such as 0.7 mm to 1.8 mm. Any of the lattice sections may have a thickness from. 1 mm to 2 mm, for example only, and which may vary along a shank, such as if a thickness is greater at a recess than at a raised support structure.

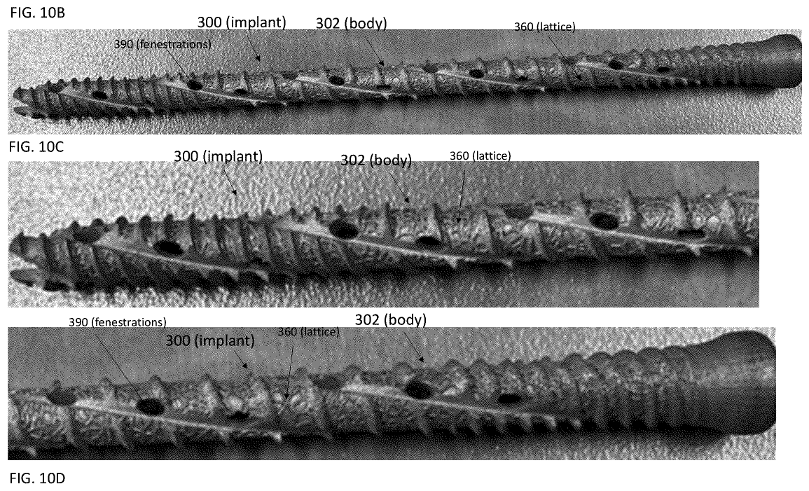

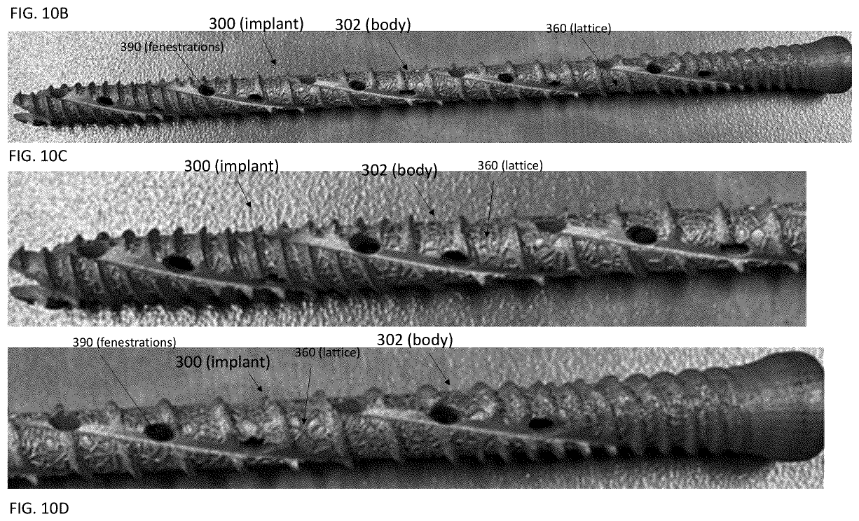

FIGS. 10A, 10B, 10C and 10D illustrate an alternative implant 300 that includes elongate body 302, and which is threaded and includes lattice sections 360 along its length, as shown. Elongate body 302 includes fenestrations 390 that extend through a shank and communicate with a central lumen, which are absent in elongate bodies 102 and 202. The absence of fenestrations in an elongate body provides greater relative strength, all other aspects of the elongate body being the same. Additionally, elongate body 302 in FIGS. 10A-10D includes helical flutes extending along its length with traditionally “valley” configurations. The valley configurations of the helical flutes do not have or create an outwardly curved surface as due some of the recessed regions described herein. Additionally, the flutes as shown in FIGS. 10A-10D do not extend as far circumferentially around the substrate (at an angle with reference to a long axis) as do some shank recesses herein.

One aspect of the disclosure includes methods of stabilizing at least a portion of a pelvis with a threaded bone implant, comprising: laterally advancing any of the threaded implants herein, through a first SI joint, through a sacral corridor (e.g., an SI corridor, an S2 corridor, or an S3 corridor, which is described in additional detail in PCT application PCT/US2023/067747), and through a contralateral SI joint, and leaving the implant implanted across both SI joints and through the sacral corridor. FIG. 2 illustrates an exemplary implant position following a lateral implantation procedure for pelvic stabilization.

One aspect of the disclosure includes methods of manufacturing any of the threaded implants herein, optionally comprising using an additive manufacturing process to create any of the threaded implants herein.

Any of the elongate bodies may be implanted with a proximal washer, examples of which are shown in FIGS. 1, 2, 3A, 6, 7, 8B.

Any of the elongate bodies herein that include an inner or central lumen may be sized to accommodate a 2.5 mm wire therethrough for delivery.

While implants herein generally include one or more raised support structures (e.g., support ribs), it is conceivable that implants herein may alternatively include shanks that do not have one or more support structures (and corresponding recessed regions), while still providing the required strength and stability. For example only, any implant herein may alternatively not include a shank with a raised support structure, but it may still include, for example, one or more single thread regions that allow additional lattice structure (compared to multi-lead regions) to promote bony integration. Additionally, implants without raised shank support structures may include interruptions in any thread to provide additional space for lattice structure, and optionally wherein at least some interruptions are disposed in single lead thread region(s).

Additionally, while implants herein generally include a plurality of thread regions, it is conceivable that implants herein may alternatively include a single thread region (e.g., a single thread with constant height) along the entire length of the elongate body while still providing the required strength and stability. For example only, any implant herein may still include, for example, one or more raised supports and corresponding recesses; lattice structure on a least a portion of a shank (e.g., in a recess and/or raised support); threads with one or more interruptions; lattice in one or more thread interruptions; and/or lattice in one or more thread interruptions that are in one or more recesses.

Additionally, while implants herein generally include one or more thread interruptions, it is conceivable that implants herein may alternatively not include any thread interruptions, while still providing the required strength and stability. For example only, any implant herein may still include, for example, a plurality of thread regions; one or more raised support structures and recesses; and/or lattice structure on a least a portion of a shank (e.g., in a recess and/or raised support).

Any structural feature from any threaded implant herein (shown, claimed or described) may be integrated and incorporated with any other suitably combinable feature from any other threaded implant herein, and vice versa.

What is claimed:

1. A threaded implant (“implant”) adapted for pelvic stabilization, comprising:
a monolithic elongate body having a distal end, a proximal end, and a length from the proximal end to the distal end,
the monolithic elongate body including,
a plurality of threaded regions including, in a proximal to distal direction, a proximal multi-lead thread region adjacent the proximal end, a proximal single lead thread region, a central multi-lead thread region, a distal single lead thread region, and a distal multi-lead thread region adjacent the distal end, the plurality of threaded regions including a plurality of threads that are disposed about a shank,
wherein each of the proximal single lead thread region, the central multi-lead thread region, and the distal single lead thread region includes a plurality of thread interruptions, and
wherein at least a portion of each of the proximal multi-lead thread region and the distal multi-lead thread region includes at least one thread that completes at least one revolution about the shank without a thread interruption,
the shank defining an inner lumen and including,
one or more raised support ribs extending along at least a portion of each of the plurality of threaded regions,
one or more recesses extending along at least a portion of each of the plurality of threaded regions, each of the one or more recesses circumferentially adjacent to one of the one or more raised support ribs,
wherein the plurality of thread interruptions in each of the proximal single lead thread region, the central multi-lead thread region, and the distal single lead thread region are disposed at the one or more recesses,
a lattice disposed about the one or more support ribs and the one or more recesses in at least a portion of each of the plurality of threaded regions,
wherein a portion of the lattice is disposed in at least one thread interruption in each of the proximal single lead thread region, the central multi-lead thread region, and the distal single lead thread region.

2. The implant of claim 1, wherein the lattice has a thickness at the one or more recesses that is greater than a lattice thickness at the one or more support ribs.

3. The implant of claim 1, wherein a plurality of thread interruptions in the central region are helically arranged and helically aligned with a shank recess.

4. The implant of claim 1, wherein the length of the elongate body is from 140 mm to 180 mm and has a diameter from 8.3 mm to 9.0 mm.

5. The implant of claim 4, wherein at least one of the proximal multi thread region has a length from 10 mm to 40 mm, the proximal single thread region has a length from 5 mm to 70 mm, the central multi thread region has a length from 10 mm to 80 mm, the distal single thread region has a length from 5 mm to 70 mm, or the distal multi thread region has a length from 10 mm to 40 mm.

6. The implant of claim 1, wherein the proximal multi-lead thread region is a dual lead thread region, the central multi-lead thread region is a dual lead thread region, and the distal multi-lead thread region is a dual lead thread region.

7. The implant of claim 1, wherein at least one of the central multi-lead thread region, the proximal single lead thread region, or the distal single lead thread region does not include a thread that makes a complete revolution about the shank.

8. The implant of claim 1, wherein at least one of the proximal multi-lead thread region or the distal multi-lead thread region does not include a thread interruption.

9. The implant of claim 1, wherein at least one of the central multi-lead thread region, the proximal single lead thread region, or the distal single lead thread region comprises at least three thread interruptions.

10. The implant of claim 1, wherein at least one of the proximal multi-lead thread region or the distal multi-lead thread region includes a thread that completes at least two revolutions without a thread interruption.

11. The implant of claim 1, wherein a thread in the proximal single lead thread region, the central multi-lead thread region, and the distal single lead thread region does not complete a revolution.

12. The implant of claim 1, wherein a plurality of thread interruptions in the proximal single lead thread region, the central multi-lead thread region, and the distal single lead thread region are helically or linearly arranged.

13. The implant of claim 1, wherein the shank, within the central multi-lead thread region, does not comprise an opening that creates radial communication between the inner lumen and an ambient space radially outside of the inner lumen.

14. The implant of claim 1, wherein the shank, within at least one of the proximal single lead thread region, or the distal single lead thread region does not comprise an opening that creates radial communication between the inner lumen and an ambient space radially outside of the inner lumen.

15. The implant of claim 1, wherein the shank, within at least one of the proximal multi-lead thread region, or the distal multi-lead thread region does not comprise an opening that creates radial communication between the inner lumen and an ambient space radially outside of the inner lumen.

16. The implant of claim 1, wherein the shank, within each of the plurality of threaded regions, does not comprise an opening that creates radial communication between the inner lumen and an ambient region radially outside of the respective threaded region.

17. The implant of claim 16, further comprising a plurality of distal cutting flutes adjacent the distal end.

18. The implant of claim 1, wherein the one or more raised support ribs comprise first and second helical support ribs, and wherein the one or more recesses comprise first and second helical recesses, each of the first and second helical recesses circumferentially adjacent to the first and second helical support ribs.

19. The implant of claim 1, wherein at least one of the one or more raised support ribs extends continuously in at least the proximal single lead thread region, the central multi-lead thread region, and the distal single lead thread region, interrupted only by threads.

20. The implant of claim 1, wherein the one or more raised support ribs comprise first and second discontinuous and axially spaced support ribs.

21. The implant of claim 1, wherein the first and second discontinuous and axially spaced support ribs are linearly or helically arranged.

22. The implant of claim 1, wherein at least one of the one or more raised support ribs has a varying height in a radial direction along at least a portion of a length of the at least one support rib.

23. The implant of claim 1, wherein at least one of the one or more raised support ribs has a varying width in a circumferential direction along at least a portion of a length of the at least one support rib.

24. The implant of claim 1, wherein the one or more recesses are curved radially outwardly.

25. The implant of claim 1, wherein each of the at least one support rib extends from 30 to 100 degrees circumferentially around shank, and wherein each of the one or more recesses extends from 30 to 100 degrees circumferentially around the shank.

26. The implant of claim 1, wherein a thread height at a location of the one or more support ribs is from 0.8 mm-1.2 mm.

27. The implant of claim 26, wherein each of the proximal single thread region, the central multi lead thread region, the distal single thread region, and the distal multi thread region include at least one thread that has the thread height of from 0.8 mm-1.2 mm.

28. The implant of claim 1, wherein the proximal multi thread region includes a first thread with a thread height that is greater than a thread height of a second thread in the proximal multi thread region.
